(19) United States
(12) Patent Application Publication
(10) Pub. No.: US 2024/0010616 A1
(43) Pub. Date: Jan. 11, 2024

(54) SIPONIMOD SALTS AND COCRYSTALS

(71) Applicant: Synthon B.V., Nijmegen (NL)

(72) Inventors: Bohumil DYMACEK, Blansko (CZ); Jiri PARTL, Blansko (CZ); Nicolas TESSON, Barcelona (ES); Montserrat TRILLA CASTANO, Barcelona (ES)

(21) Appl. No.: 18/246,566

(22) PCT Filed: Sep. 24, 2021
(86) PCT No.: PCT/EP2021/076406

§ 371 Date: Mar. 24, 2023

(30) Foreign Application Priority Data
Sep. 25, 2020 (EP) 20198482.0
Feb. 8, 2021 (EP) 21155774.9

(51) Int. Cl. C07D 205/04; C07C 55/14

(52) U.S. Cl. CPC C07D 205/04; C07C 55/14; C07B 2200/13

(57) ABSTRACT

The presented invention relates to solid forms of Siponimod adipic acid salt and cocrystal.

### BACKGROUND OF THE PRESENT INVENTION

The present invention relates to salt or cocrystal of the compound Siponimod with adipic acid.

Siponimod, 1-{4-[1-(4-cyclohexyl-3-trifluoromethyl-benzyloxyimino)-ethyl]-2-ethyl-benzyl}-azetidine-3-carboxylic acid of formula (I),

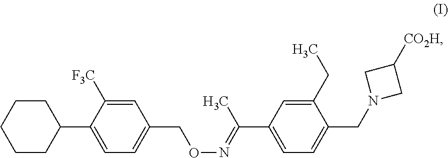 [SMILES: CCc1cc(/C(C)=N/OCc2ccc(C3CCCCC3)c(C(F)(F)F)c2)ccc1CN1CC(C(=O)O)C1]

is a lysophospholipid EDG1 (S1P1) receptor ligand that is useful for treatment of immunological disorders. Fumaric acid salt of siponimod is now ongoing pre-registration for use in the treatment secondary progressive multiple sclerosis.

Siponimod was first disclosed in WO2004/103306 by Novartis. Hemifumarate salt of Siponimod (i.e. ratio Siponimod:Fumaric acid is 1:0.5) and solid forms A, B, C, D and E of the hemifurate salt are disclosed in WO2010/080409 by Novartis. HCl salt, malate salt, oxalate salt, tartrate salt and their crystalline forms are disclosed in WO2010/080455 by Novartis.

The prior art fumarate salt prepared according to WO2010/080409 forms fine crystals that are prone to charging. The processing of such crystals into final forms, for example tablets, is complicated. On the other hand, the solid forms of presented invention form bigger crystals that are not prone to charging and therefore having improved processing properties.

### SUMMARY OF THE INVENTION

The presented invention relates to solid forms of Siponimod adipic acid (hexanedioic acid) salt and cocrystal.

The presented invention relates to a solid form of cocrystal of Siponimod with adipic acid (2:1), Form 11, characterized by XRPD pattern having 2θ values 6.7°, 12.7° and 13.5° 2θ (±0.2 degrees 2θ).

The presented invention also relates to a process for preparation of the solid form of cocrystal of Siponimod with adipic acid, Form 11, the process comprising:
a. Dissolving Siponimod in ethylacetate;
b. Adding adipic acid;
c. Isolating the solid form.

The presented invention further relates to a solid form of Siponimod adipic acid salt (1:2), Form Q′, characterized by XRPD pattern having 2θ values 5.5°, 17.5° and 18.8° 2θ (±0.2 degrees 2θ).

The presented invention also relates to a process for preparation of the solid form of Siponimod adipic acid salt, Form Q′, comprising:
a. Dissolving Siponimod and adipic acid in methyl tert-butyl ether;
b. Isolating a solid form;
c. Contacting the solid with humidity.

The presented invention relates to a solid form of Siponimod adipic acid salt (1:2), Form Q, characterized by XRPD pattern having 2θ values 5.3°, 18.10 and 20.3° 2θ (±0.2 degrees 2θ).

The presented invention also relates to a process for preparation of the solid form of Siponimod adipic acid salt, Form Q, comprising:
a. Dissolving Siponimod and adipic acid in methyl tert-butyl ether;
b. Isolating the solid form.

The presented invention also relates to a solid form of Siponimod adipic acid salt (1:1), Form P, characterized by XRPD pattern having 2θ values 5.6°, 7.5° and 19.2° 2θ (±0.2 degrees 2θ). The solid form of Siponimod adipic acid salt, Form P, can be prepared by a process comprising:
a. Dissolving Siponimod and adipic acid in methanol and n-heptane;
b. Isolating the solid form.

The presented invention also relates to a solid form of Siponimod adipic acid salt (1:2), Form 1, characterized by XRPD pattern having 2θ values 15.5°, 18.0°, 19.3 and 21.5° 2θ (±0.2 degrees 2θ). The solid form of Siponimod adipic acid salt, Form 1, can be prepared by a process comprising:
a. Suspending Siponimod and adipic acid in methyl tert-butyl ether;
b. Isolating the solid form.

The presented invention further relates to a solid form of Siponimod adipic acid salt (1:0.6), Form 2, characterized by XRPD pattern having 2θ values 4.0°, 7.4°, 17.8° and 19.3° 2θ (±0.2 degrees 2θ). The solid form of Siponimod adipic acid, Form 2, can be prepared by a process comprising:
a. Dissolving Siponimod and adipic acid in methanol;
b. Adding n-heptane;
c. Isolating the solid form.

The prior art fumarate salt prepared according to WO2010/080409 forms fine crystals that are prone to charging. The processing of such crystals into final forms, for example tablets, is complicated. On the other hand, the solid forms of presented invention form bigger crystals that are not prone to charging and therefore having improved processing properties. Also the water activity of Form 11 is better comparing to prior art forms.

### BRIEF DESCRIPTION OF THE DRAWINGS

FIG. 1: XRPD pattern of solid Form Q of Siponimod adipic acid salt (1:2) prepared according to Example 1

FIG. 2: DSC pattern of solid Form Q of Siponimod adipic acid salt (1:2) prepared according to Example 1

FIG. 3: NMR pattern of solid Form Q of Siponimod adipic acid salt (1:2) prepared according to Example 1

FIG. 4: XRPD pattern of solid Form Q′ of Siponimod adipic acid salt (1:2) prepared according to Example 1

FIG. 5: DSC pattern of solid Form Q′ of Siponimod adipic acid salt (1:2) prepared according to Example 1

FIG. 6: NMR pattern of solid Form Q′ of Siponimod adipic acid salt (1:2) prepared according to Example 1

FIG. 7: XRPD pattern of solid Form P of Siponimod adipic acid salt (1:1) prepared according to Example 2

FIG. 8: DSC pattern of solid Form P of Siponimod adipic acid salt (1:1) prepared according to Example 2

FIG. 9: NMR pattern of solid Form P of Siponimod adipic acid salt (1:1) prepared according to Example 2

FIG. 10: XRPD pattern of solid Form 1 of Siponimod adipic acid salt (1:2) prepared according to Example 3

FIG. 11: DSC pattern of solid Form 1 of Siponimod adipic acid salt (1:2) prepared according to Example 3

FIG. 12: NMR pattern of solid Form 1 of Siponimod adipic acid salt (1:2) prepared according to Example 3

FIG. 13: XRPD pattern of solid Form 2 of Siponimod adipic acid salt (1:0.6) prepared according to Example 4

FIG. 14: DSC pattern of solid Form 2 of Siponimod adipic acid salt (1:0.6) prepared according to Example 4

FIG. 15: NMR pattern of solid Form 2 of Siponimod adipic acid salt (1:0.6) prepared according to Example 4

FIG. 16: XRPD pattern of solid Form 4 of Siponimod adipic acid salt (1:2) prepared according to Example 5

FIG. 17: Crystals of solid Form Q′ prepared according to Example 1

FIG. 18: Crystals of solid Form Q prepared according to Example 1

FIG. 19: Crystals of solid Form P prepared according to Example 2

FIG. 20: Crystals of solid Form 1 prepared according to Example 3

FIG. 21: Crystals of solid Form 2 prepared according to Example 4

FIG. 22: Crystals of solid form of Siponimod fumarate salt prepared according to WO2010/080409

FIG. 23: XRPD pattern of solid Form 11 of cocrystal of Siponimod with adipic acid (2:1) prepared according to Example 7 or Example 8 or Example 9 or Example 10.

FIG. 24: DSC pattern of solid Form 11 of cocrystal of Siponimod with adipic acid (2:1) prepared according to Example 7 or Example 8 or Example 9 or Example 10.

FIG. 25: Crystals of solid Form 11 of cocrystal of Siponimod with adipic acid (2:1) prepared according to Example 8.

FIG. 26: Crystals of solid Form 11 of cocrystal of Siponimod with adipic acid (2:1) prepared according to Example 7.

FIG. 27: Crystals of solid Form 11 of cocrystal of Siponimod with adipic acid (2:1) prepared according to Example 9.

FIG. 28: Crystals of solid Form A of Siponimod fumarate prepared according to WO2010/080409.

FIG. 29: NMR pattern of solid Form 11 of cocrystal of Siponimod with adipic acid (2:1) prepared according to Example 7 or Example 8 or Example 9.

### DETAILED DESCRIPTION OF THE INVENTION

The presented invention relates to Siponimod adipic acid salt or cocrystal and solid forms thereof. A cocrystal can be defined as a binary compound where two components (co-formers) are in a solid state connected via non-ionic intermolecular bonds.

The presented invention relates to a solid form of cocrystal of Siponimod with adipic acid (2:1), Form 11. The solid form, Form 11, can be characterized by XRPD pattern having 2θ values 6.7°, 12.7° and 13.5° 2θ (±0.2 degrees 2θ). The Form 11 can be also characterized by XRPD pattern having 2θ values 6.7°, 7.5°, 12.7°, 13.5°, 16.4° and 18.3° 2θ (±0.2 degrees 2θ). The Form 11 can be further characterized by XRPD pattern described in following table:

| Angle 2θ | Intensity % |
|---|---|
| 6.7 | 43.3 |
| 7.5 | 43.2 |
| 11.0 | 40.9 |
| 12.3 | 45.4 |
| 12.7 | 50.0 |
| 13.3 | 6.6 |
| 13.5 | 100.0 |
| 14.5 | 40.0 |
| 14.6 | 23.7 |
| 15.2 | 3.1 |
| 16.4 | 46.4 |
| 17.0 | 24.1 |
| 17.2 | 15.9 |
| 17.5 | 28.8 |
| 18.3 | 56.4 |
| 18.7 | 11.2 |
| 18.9 | 34.6 |
| 19.8 | 12.1 |
| 20.0 | 31.2 |
| 20.3 | 18.9 |
| 21.7 | 11.8 |
| 22.0 | 38.2 |
| 22.2 | 35.5 |
| 22.9 | 20.8 |
| 23.2 | 26.0 |
| 24.0 | 6.8 |
| 24.3 | 10.6 |
| 24.5 | 11.5 |
| 24.7 | 17.8 |
| 25.1 | 9.9 |
| 25.5 | 9.6 |
| 25.9 | 8.0 |
| 26.5 | 10.1 |
| 27.2 | 7.2 |
| 28.0 | 6.1 |
| 28.5 | 6.0 |
| 28.7 | 6.5 |
| 29.3 | 4.6 |
| 29.4 | 4.9 |
| 29.5 | 4.0 |
| 30.5 | 4.2 |
| 30.6 | 4.8 |
| 30.7 | 6.1 |
| 31.0 | 6.2 |
| 31.8 | 9.0 |
| 32.2 | 5.2 |
| 32.6 | 3.8 |
| 32.7 | 3.9 |
| 33.1 | 5.6 |
| 33.2 | 5.3 |
| 34.5 | 3.5 |

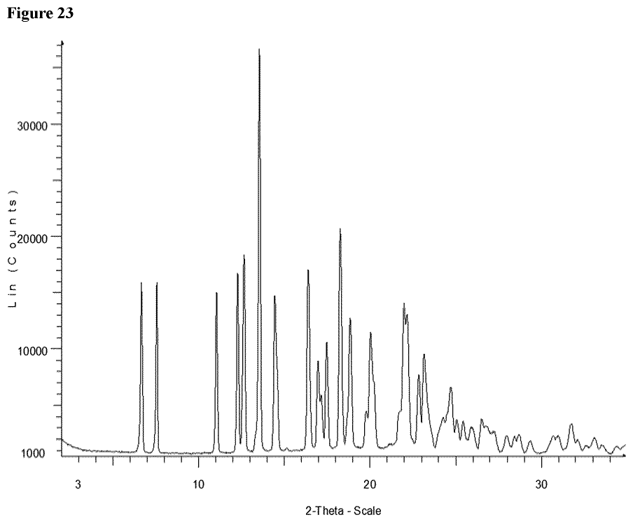

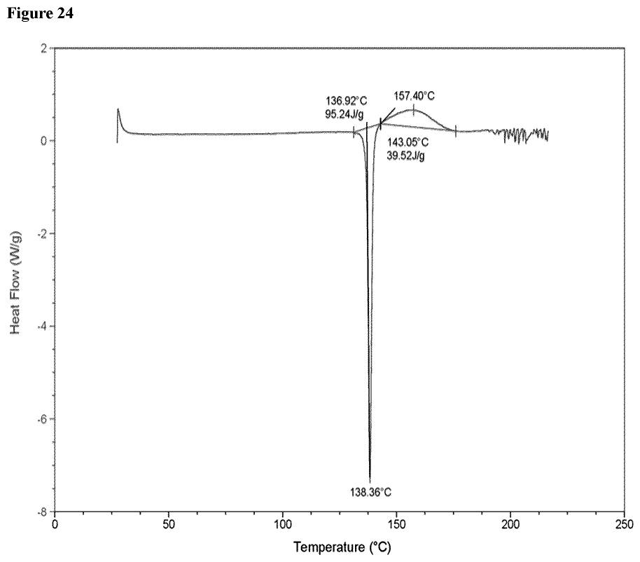

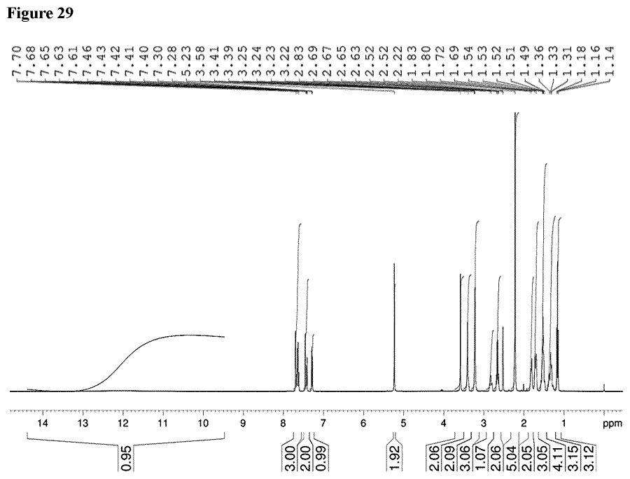

The Form 11 can be also characterized by XPRD pattern depicted in FIG. 23 or DSC pattern depicted in FIG. 24 or NMR pattern depicted in FIG. 29.

The solid Form 11 of cocrystal of Siponimod with adipic acid (2:1), can be prepared by a process comprising:
a. Dissolving Siponimod in ethylacetate;
b. Adding adipic acid;
c. Isolating the solid form.

Siponimod is dissolved in ethylacetate. The ethylacetate can optionally contain water up to 3% (vol %). The concentration of Siponimod in the ethylacetate can be between 0.02 g/ml and 0.65 g/ml. To the solution adipic acid is added. The molar ratio between Siponimod and adipic acid can be between 1:0.5 and 1:1.1. The mixture is heated to a temperature between 55° C.-75° C. and stirred at this temperature for between 10 and 60 minutes. The mixture is cooled to a temperature between 35° C. and 45° C. The mixture can be optionally seeded with seeds of cocrystal of Siponimod with adipic acid Form 11. The seeds can be prepared for example by the procedure disclosed in Example 7. The mixture is stirred at this temperature for between 1 and 5 hours. The mixture is cooled to between 15° C. and 25° C. in the course of between 1.5 and 3 hours. The mixture is stirred at this temperature for between 2 and 6 hours. The solid is filtered off, optionally washed and dried to obtain solid Form 11 of cocrystal of Siponimod with adipic acid (2:1).

The presented invention also relates solid forms of Siponimod adipic acid salt (1:2), Forms Q and Q′, and a process for preparation thereof.

Siponimod adipic acid salt (1:2), Form Q′, can be characterized by XRPD pattern having 2θ values 5.5°, 17.5° and 18.8° 2θ (±0.2 degrees 2θ). The Form Q′ can be also characterized by XRPD pattern having 2θ values 5.5°, 7.6°, 16.5°, 17.5° and 18.8° 2θ (±0.2 degrees 2θ). The Form Q′ can be further characterized by XRPD pattern described in the following Table:

| Angle 2θ | Intensity % |
|---|---|
| 5.5 | 100.0 |
| 7.2 | 10.3 |
| 7.6 | 30.9 |
| 8.9 | 12.0 |
| 9.8 | 10.6 |
| 10.5 | 12.5 |
| 10.8 | 15.9 |
| 11.0 | 17.9 |
| 11.6 | 9.5 |
| 12.6 | 16.7 |
| 12.8 | 17.3 |
| 13.6 | 17.8 |
| 16.5 | 22.4 |
| 17.5 | 54.4 |
| 18.8 | 54.2 |
| 19.7 | 57.7 |
| 20.7 | 20.8 |
| 21.5 | 33.2 |
| 22.1 | 24.3 |
| 23.0 | 19.9 |
| 23.7 | 16.7 |
| 25.2 | 11.8 |
| 25.8 | 12.5 |
| 26.5 | 10.8 |
| 27.0 | 12.7 |
| 27.3 | 15.5 |
| 27.8 | 12.4 |
| 30.2 | 6.6 |
| 31.1 | 6.0 |

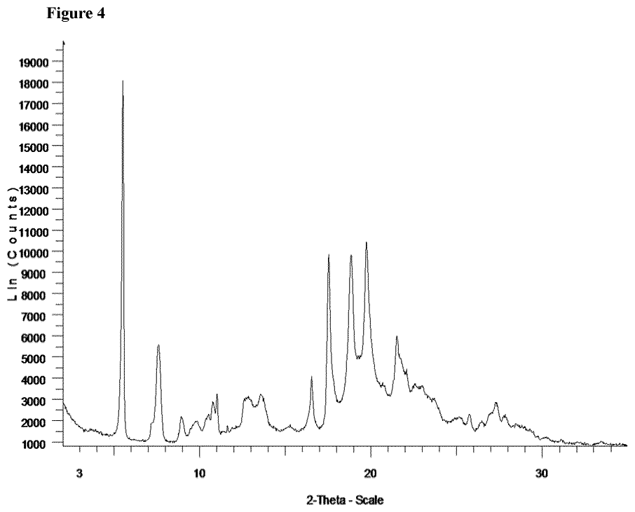

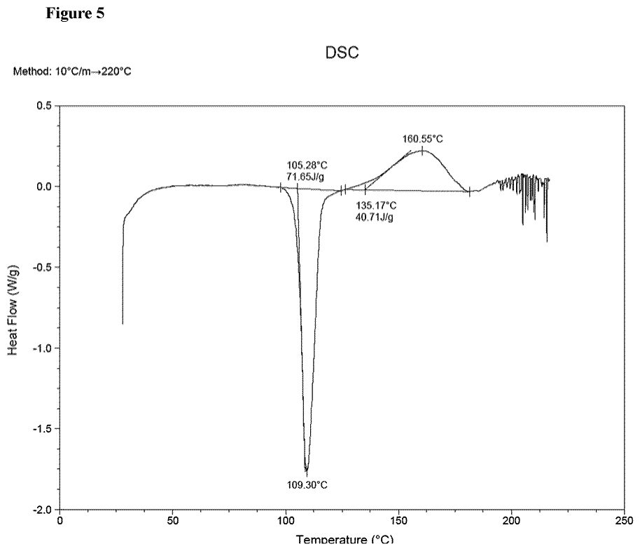

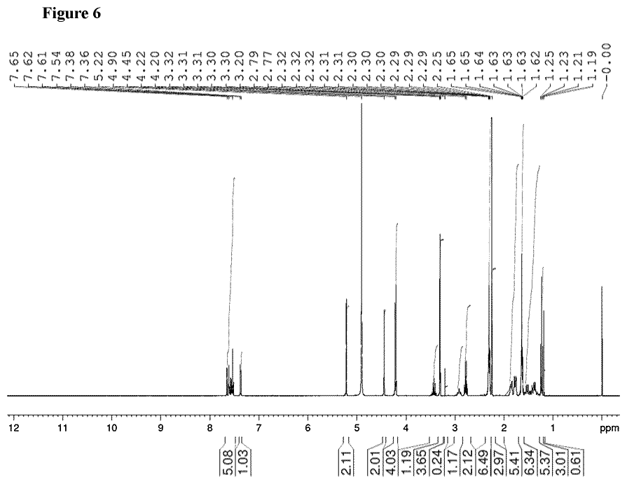

The Form Q′ can be also characterized by XPRD pattern depicted in FIG. 4 or DSC pattern depicted in FIG. 5 or NMR pattern depicted in FIG. 6.

Siponimod adipic acid salt (1:2), Form Q, can be characterized by XRPD pattern having 2θ values 5.3°, 18.1° and 20.3° 2θ (±0.2 degrees 2θ). Form Q can be also characterized by XRPD pattern having 2θ values 5.3°, 12.8°, 18.1°, 19.6° and 20.3° 2θ (±0.2 degrees 2θ). The Form Q can be further characterized by XRPD pattern described in the following Table:

| Angle 2θ | Intensity % |
|---|---|
| 3.7 | 24.3 |
| 5.3 | 100.0 |
| 7.7 | 39.3 |
| 8.6 | 11.5 |
| 8.8 | 11.1 |
| 10.4 | 11.4 |
| 10.7 | 16.8 |
| 11.5 | 10.8 |
| 12.8 | 42.8 |
| 13.5 | 19.1 |
| 14.0 | 14.6 |
| 14.9 | 17.9 |
| 16.1 | 16.6 |
| 17.3 | 74.1 |
| 18.1 | 56.4 |
| 18.4 | 39.5 |
| 18.9 | 44.2 |
| 19.6 | 47.4 |
| 20.3 | 76.0 |
| 21.5 | 42.4 |
| 21.9 | 30.6 |
| 23.8 | 28.8 |
| 24.6 | 21.0 |
| 25.7 | 13.2 |
| 26.8 | 17.2 |
| 27.6 | 15.2 |
| 28.3 | 15.3 |
| 28.7 | 13.0 |
| 29.3 | 9.4 |
| 30.7 | 9.5 |
| 31.1 | 7.1 |
| 32.3 | 7.1 |

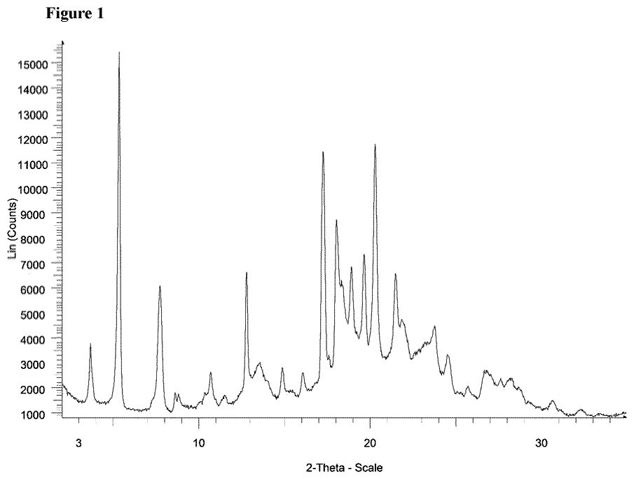

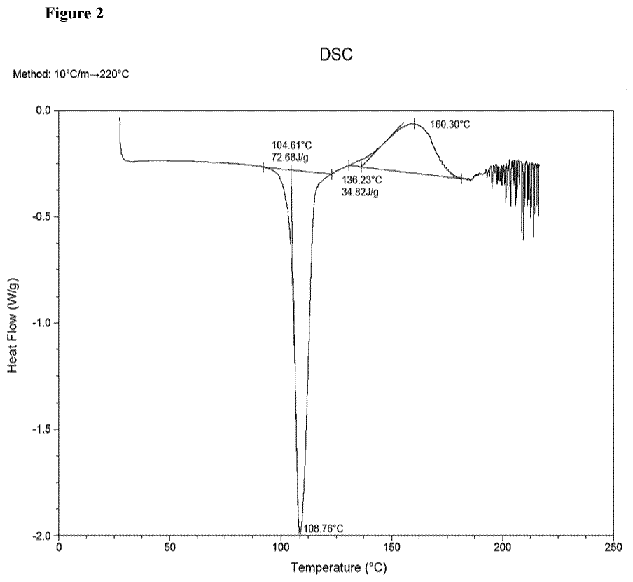

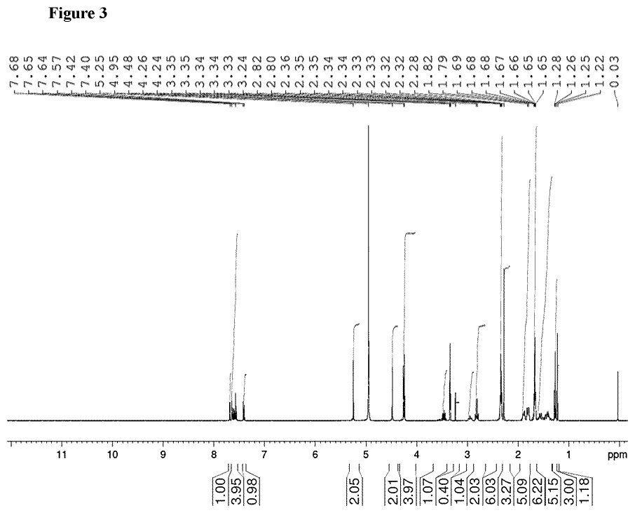

The Form Q can be also characterized by XPRD pattern depicted in FIG. 1 or DSC pattern depicted in FIG. 2 or NMR pattern depicted in FIG. 3.

The solid Form Q′ of a salt of Siponimod with adipic acid (1:2) can be prepared by a process comprising:
a. Dissolving Siponimod and adipic acid in methyl tert-butyl ether;
b. Isolating a solid form;
c. Contacting the solid form with humidity to obtain Form Q′.

The solid form isolated in step b. is preferably solid form Q of a salt of Siponimod with adipic acid (1:2). Siponimod and adipic acid are dissolved in methyl-tert butyl ether. The concentration of Siponimod in the methyl-tert butyl ether can be between 0.02 g/ml and 0.06 g/ml. The concentration of adipic acid in the methyl-tert butyl ether can be between 0.015 and 0.05 g/ml. The molar ratio between Siponimod and adipic acid can be between 1:2 and 1:2.2. The mixture is left stayed at a temperature between 20° C. and 25° C. to evaporate the solvent until final volume between 25% and 30% (vol/vol) of the starting volume to obtain a suspension. The suspension is filtered. The obtained solid can be dried to obtain Siponimod adipic acid salt (1:2), Form Q. Obtained solid Form Q is exposed to the temperature between 40° C. and 60° C. and the humidity between 80% and 100% RH for between 10 and 15 hours to provide Siponimod adipic acid salt (1:2), Form Q′.

The invention also relates to a solid form of Siponimod adipic acid salt (1:1), Form P, characterized by XRPD pattern having 2θ values 5.6°, 7.5° and 19.2° 2θ (±0.2 degrees 2θ). The Form P can be also characterized by XRPD pattern having 2θ values 5.6°, 7.5°, 13.8°, 17.7° and 19.2° 2θ (±0.2 degrees 2θ). The Form P can be further characterized by XRPD pattern described in the following Table:

| Angle 2θ | Intensity % |
|---|---|
| 3.9 | 36.2 |
| 5.6 | 87.1 |
| 7.5 | 70.4 |
| 8.0 | 17.3 |
| 9.5 | 18.2 |
| 10.5 | 40.5 |
| 10.7 | 39.0 |
| 11.3 | 29.7 |
| 11.7 | 25.2 |
| 12.4 | 35.1 |
| 13.0 | 36.2 |
| 13.5 | 32.3 |
| 13.8 | 55.0 |
| 15.0 | 19.6 |
| 15.5 | 26.3 |
| 15.7 | 33.3 |
| 16.1 | 25.0 |
| 16.6 | 27.0 |
| 17.0 | 30.9 |
| 17.7 | 67.6 |
| 18.4 | 46.0 |
| 19.2 | 100.0 |
| 19.9 | 84.0 |
| 20.4 | 50.2 |
| 20.9 | 65.9 |
| 21.5 | 53.8 |
| 22.1 | 60.6 |
| 22.8 | 49.6 |
| 23.1 | 35.5 |
| 23.7 | 39.4 |
| 24.5 | 27.9 |
| 25.0 | 22.2 |
| 25.4 | 23.0 |
| 25.8 | 25.0 |
| 26.0 | 26.4 |
| 27.1 | 29.2 |
| 28.2 | 19.3 |
| 28.5 | 23.1 |
| 29.6 | 16.6 |
| 30.3 | 14.1 |
| 31.2 | 13.4 |
| 32.5 | 10.9 |
| 33.7 | 10.9 |
| 34.3 | 10.4 |

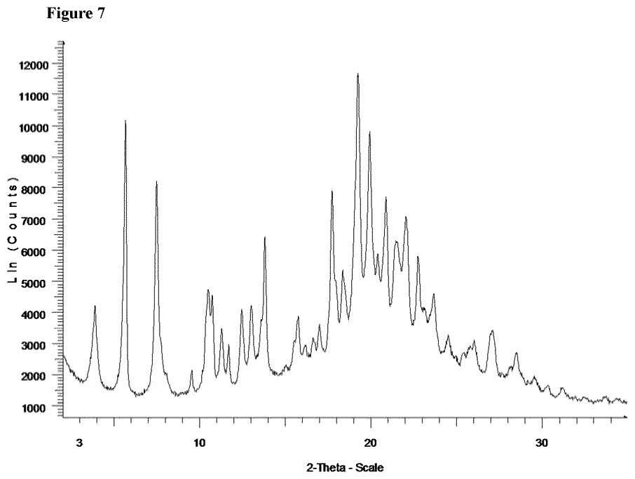

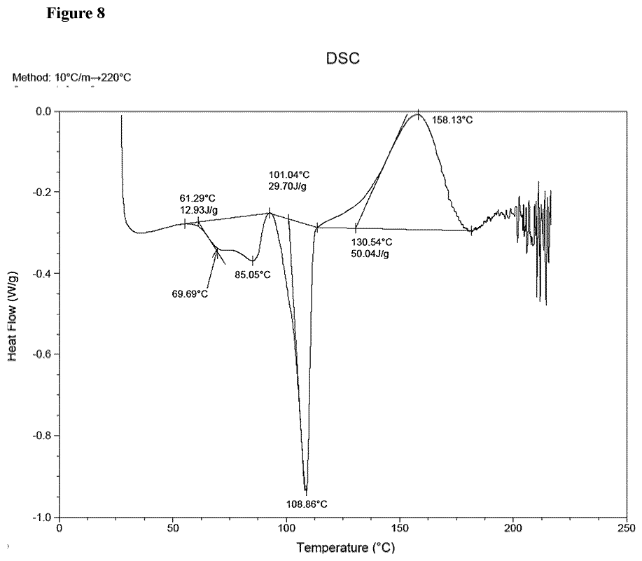

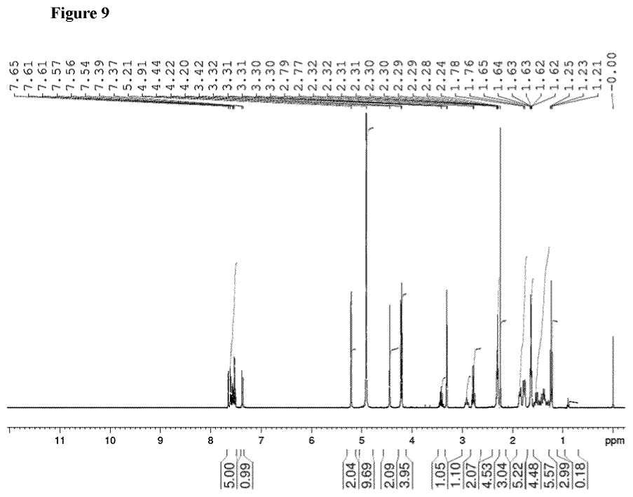

The Form P can be also characterized by XPRD pattern depicted in FIG. 7 or DSC pattern depicted in FIG. 8 or NMR pattern depicted in FIG. 9.

The solid form of Siponimod adipic acid (1:1), Form P, can be prepared by a process comprising:
a. Dissolving Siponimod and adipic acid in methanol and n-heptane;
b. Isolating the solid form.

The volume ratio between methanol and n-heptane can be between 1:6 and 1:8. Siponimod and adipic acid are dissolved in methanol. The concentration of Siponimod in methanol can be between 0.2 g/ml and 0.5 g/ml. The concentration of adipic acid in methanol can be between 0.07 g/ml and 0.15 g/ml. To the mixture n-heptane is added. From the mixture, approximately 10 drops are taking away and left crystallize for 3 days at 20-25° C. on a closed Petri dish to obtain crystals. The crystals are added to the previously prepared solution of Siponimod and adipic acid in methanol and n-heptane. The mixture is then stirred at 20-25° C. for between 1 and 10 hours to obtain a suspension. The suspension is filtered and obtained solid Form P can be optionally dried.

The presented invention further relates to a solid form of Siponimod adipic acid salt (1:2), Form 1, characterized by XRPD pattern having 2θ values 15.5°, 18.0°, 19.3° and 21.5° 2θ (±0.2 degrees 2θ). The Form 1 can be also characterized by XRPD pattern having 2θ values 9.9°, 14.3°, 15.5°, 18.0°, 19.3°, 21.0° and 21.5° 2θ (±0.2 degrees 2θ). The Form 1 can be further characterized by XRPD pattern described in following Table:

| Angle 2θ | Intensity % |
|---|---|
| 3.8 | 12.0 |
| 7.7 | 9.8 |
| 9.0 | 3.2 |
| 9.9 | 18.2 |
| 11.6 | 12.6 |
| 12.1 | 8.9 |
| 13.0 | 17.4 |
| 14.3 | 18.4 |
| 14.9 | 5.7 |
| 15.5 | 38.2 |
| 15.9 | 6.2 |
| 16.5 | 3.8 |
| 17.3 | 12.2 |
| 18.0 | 23.3 |
| 18.2 | 16.3 |
| 18.6 | 11.4 |
| 19.0 | 7.3 |
| 19.3 | 25.1 |
| 19.8 | 7.7 |
| 20.2 | 9.0 |
| 20.6 | 7.5 |
| 21.0 | 20.2 |
| 21.5 | 100.0 |
| 21.8 | 17.0 |
| 22.1 | 12.2 |
| 22.6 | 8.8 |
| 23.2 | 15.0 |
| 23.9 | 7.4 |
| 24.6 | 9.4 |
| 25.3 | 18.0 |
| 25.8 | 30.9 |
| 26.9 | 8.6 |
| 29.7 | 4.7 |
| 30.0 | 4.9 |
| 30.2 | 4.3 |
| 31.2 | 14.8 |
| 32.1 | 3.8 |
| 33.5 | 3.8 |
| 34.2 | 2.6 |

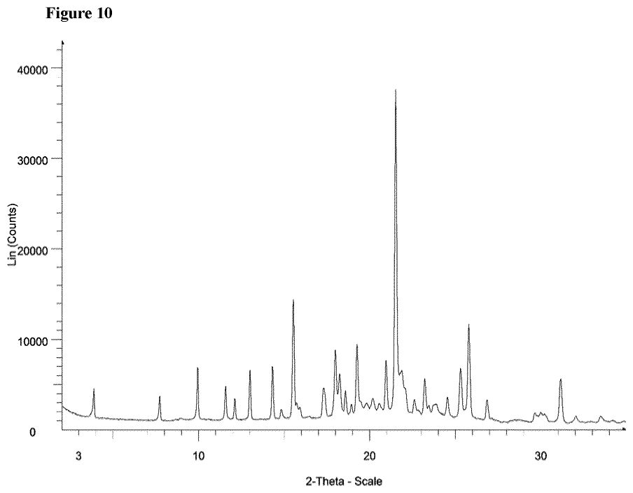

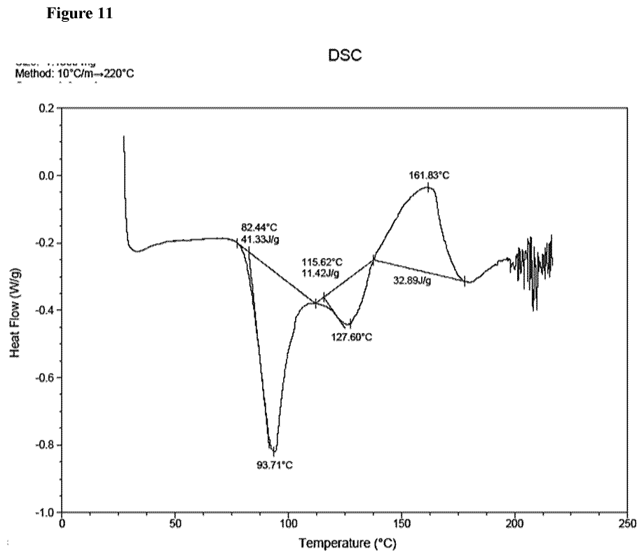

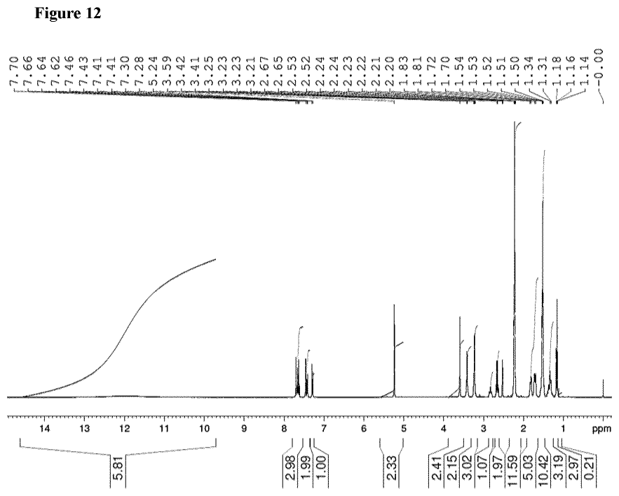

The Form 1 can be also characterized by XPRD pattern depicted in FIG. 10 or DSC pattern depicted in FIG. 11 or NMR pattern depicted in FIG. 12.

The solid form of Siponimod adipic acid (1:2), Form 1, can be prepared by a process comprising:
a. Suspending Siponimod and adipic acid in methyl tert-butyl ether;
b. Isolating the solid form.

The concentration of Siponimod in methyl tert-butyl ether can be between 0.07 g/ml and 0.15 g/ml. The concentration of adipic acid in methyl tert-butyl ether can be between 0.04 g/ml and 0.07 g/ml. The molar ratio between Siponimod and adipic acid can be between 1:2 and 1:2.2. Siponimod and adipic acid are contacted with methyl tert-butyl ether. The suspension is stirred at a temperature between 20° C. and 25° C. for between 10 and 20 hours. The suspension is filtered, obtain solid can be optionally dried to obtain solid Form 1 of Siponimod adipic acid (1:2) salt.

The presented invention further relates to a solid form of Siponimod adipic acid salt (1:0.6), Form 2, characterized by XRPD pattern having 2θ values 4.0°, 7.4°, 17.8° and 19.3° 2θ (±0.2 degrees 2θ). The Form 2 can be also characterized by XRPD pattern having 2θ values 4.0°, 5.7°, 7.4°, 17.8°, 19.3°, 20.0° and 21.0° 2θ (±0.2 degrees 2θ). The Form 2 can be further characterized by XRPD pattern described in following Table:

| Angle 2θ | Intensity % |
|---|---|
| 4.0 | 64.6 |
| 5.7 | 74.8 |
| 7.4 | 100.0 |
| 7.8 | 13.4 |
| 8.3 | 11.6 |
| 9.0 | 9.1 |
| 9.5 | 23.1 |
| 10.2 | 21.2 |
| 10.5 | 31.3 |
| 11.3 | 12.4 |
| 12.4 | 24.6 |
| 13.0 | 15.7 |
| 13.2 | 26.1 |
| 13.5 | 20.2 |
| 14.0 | 21.6 |
| 16.0 | 17.4 |
| 16.4 | 17.4 |
| 17.1 | 21.5 |
| 17.8 | 48.1 |
| 18.5 | 19.5 |
| 19.3 | 61.2 |
| 20.0 | 43.6 |
| 21.0 | 49.9 |
| 21.4 | 26.5 |
| 21.7 | 22.2 |
| 22.3 | 35.9 |
| 22.7 | 27.9 |
| 23.7 | 22.0 |
| 24.7 | 17.4 |
| 25.2 | 14.4 |
| 26.2 | 13.1 |
| 27.2 | 22.2 |
| 28.4 | 13.5 |
| 29.6 | 9.8 |
| 31.3 | 7.9 |
| 33.8 | 5.9 |

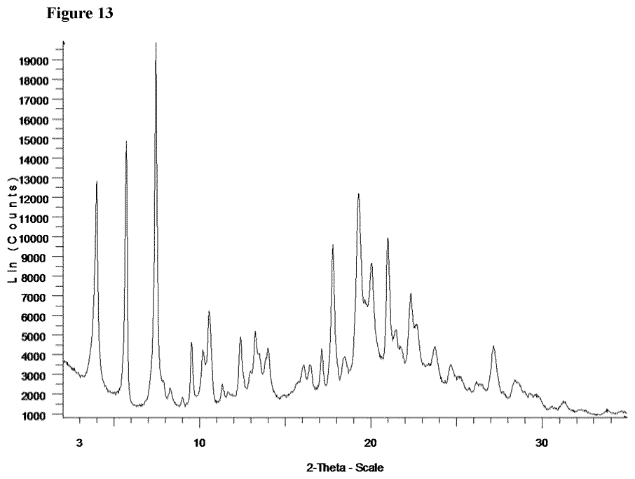

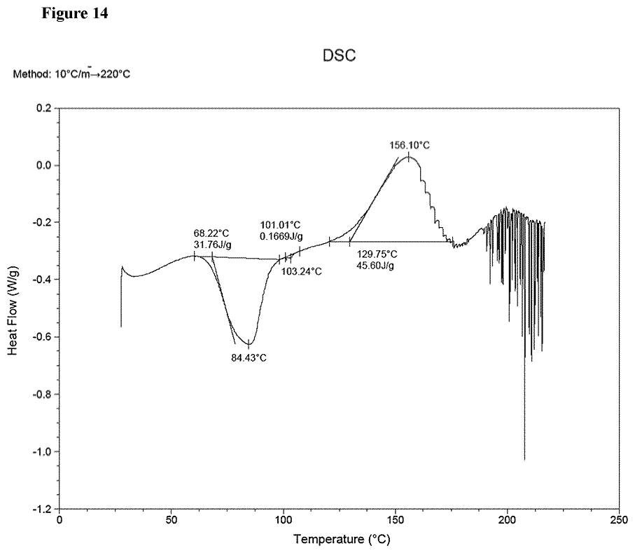

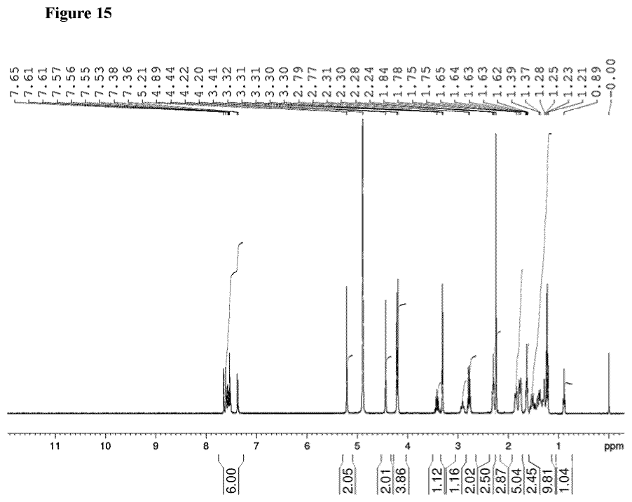

The Form 2 can be also characterized by XPRD pattern depicted in FIG. 13 or DSC pattern depicted on FIG. 14 or NMR pattern depicted on FIG. 15.

The solid Form 2 can be prepared by a process comprising:
a. Dissolving Siponimod and adipic acid in methanol;
b. Adding n-heptane;
c. Isolating the solid form.

The concentration of Siponimod in methanol can be between 0.3 and 0.6 g/ml. The concentration of adipic acid in methanol can be between 0.04 g/ml and 0.08 g/ml. The molar ratio between Siponimod and adipic acid can be between 1:0.5 and 1:0.7. Siponimod and adipic acid are dissolved in methanol. To the mixture n-heptane is added. The volume ratio between n-heptane and methanol can be between 7:1 and 9:1. The mixture is heated to a temperature between 35° C. and 50° C. to obtain a solution. The mixture is then cooled to a temperature between −5° C. and 5° C. and stirred at this temperature for between 15 and 120 minutes to obtain a suspension. Obtained solid can be isolated by any suitable technique for example using filtration and optionally dried.

The presented invention further relates to a solid form of Siponimod adipic acid salt (1:2), Form 4, characterized by XRPD pattern having 2θ values 12.6°, 18.4°, 21.5° and 23.16° 2θ (±0.2 degrees 2θ). The Form 4 can be also characterized by XRPD pattern having 2θ values 12.6°, 13.0°, 15.3°, 18.4°, 21.5° and 23.2° 2θ (±0.2 degrees 2θ). The Form 4 can be further characterized by XRPD pattern described in following Table:

| Angle 2θ | Intensity % |
|---|---|
| 3.8 | 6.3 |
| 7.5 | 17.5 |
| 8.8 | 9.4 |
| 10.6 | 11.4 |
| 11.3 | 8.3 |
| 11.6 | 7.9 |
| 12.6 | 48.2 |
| 13.0 | 18.8 |
| 13.3 | 4.3 |
| 14.5 | 11.3 |
| 15.1 | 6.9 |
| 15.3 | 15.9 |
| 15.5 | 13.2 |
| 16.0 | 11.0 |
| 16.6 | 21.5 |
| 17.0 | 13.6 |
| 17.6 | 11.5 |
| 18.1 | 13.7 |
| 18.4 | 44.9 |
| 18.9 | 10.4 |
| 19.2 | 10.0 |
| 20.1 | 10.6 |
| 20.5 | 10.3 |
| 20.9 | 13.7 |
| 21.5 | 100.0 |
| 22.7 | 20.8 |
| 23.2 | 41.5 |
| 24.0 | 8.1 |
| 24.7 | 6.9 |
| 25.1 | 8.4 |
| 25.3 | 19.2 |
| 25.8 | 28.6 |
| 26.6 | 6.8 |
| 26.9 | 13.4 |
| 27.0 | 11.2 |
| 28.4 | 4.0 |
| 29.8 | 3.2 |
| 30.3 | 4.0 |
| 30.8 | 4.9 |
| 31.2 | 14.3 |
| 32.1 | 3.9 |
| 33.6 | 4.2 |

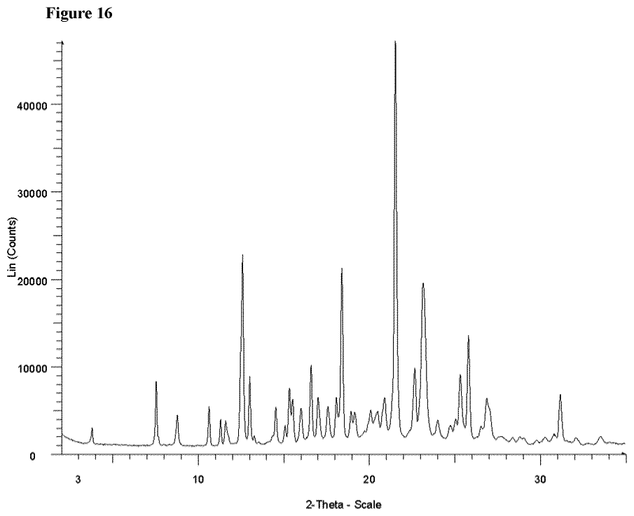

The Form 4 can be also characterized by XPRD pattern depicted in FIG. 16.

The solid form of Siponimod adipic acid salt (1:2), Form 4, can be prepared by a process comprising exposing Siponimod adipic acid salt (1:2), Form 1, to a temperature between 35° C. and 50° C., humidity between 60% and 100% of relative humidity for between 1 and 6 months.

### EXAMPLES

XRPD spectrum of solid compounds was obtained using the following measurement conditions:

Panalytical Empyrean diffractometer with Θ/2Θ geometry (transmition mode), equipped with a PixCell 3D detector

| Start angle (2θ): | 2.0° | |
|---|---|---|
| End angle (2θ): | 35.0° | |
| Step size: | 0.026° | |
| Scan speed: | 0.0955°/seconds | |
| Radiation type: | Cu | |
| Radiation wavelengths: | 1.5406 Å (Kα1), primary monochromator used | |
| Divergence slit: | ½° | |
| Antiscatter slit: | ½° | |
| Soller slit: | 0.02 | rad |
| Detector slit: | 7.5 | mm |
| Rotation speed: | 30 | rpm |

DCS patterns were obtained using the following conditions: 10° C./min->250° C.
Pictures of crystals were obtained by SEM Jeol JCM-6000.
Nuclear magnetic resonance spectroscopy (NMR) was performed using Avance III 400 MHz NMR spectrometer.

### Example 1: Preparation of Solid Forms of Siponimod Adipic Acid Salt (1:2), Forms Q and Q′

0.64 g of Siponimod and 0.36 g of adipic acid were dissolved in 18 ml of methyl tert-butyl ether (MTBE). The solution was filtered and left stayed at a temperature between 20° C. and 25° C. to evaporate the solvent until final volume of approximately 5 ml to obtain a suspension. The suspension was filtered. The filter cake 1 was dried (25° C./1 h/vacuum) to provide 0.62 g of Siponimod adipic acid (1:2) salt, Form Q. XRPD pattern of obtained solid corresponds to XRPD pattern depicted in FIG. 1, DSC pattern of obtained solid corresponds to DSC pattern depicted in FIG. 2 and NMR pattern of obtained solid corresponds to NMR pattern depicted in FIG. 3. The obtained solid was exposed to 50° C. and 100% of relative humidity for 12 hours to obtain 0.62 g of Siponimod adipic acid (1:2) salt, Form Q′. XRPD pattern of obtained solid corresponds to XRPD pattern depicted in FIG. 4, DSC pattern of obtained solid corresponds to DSC pattern depicted in FIG. 5 and NMR pattern of obtained solid corresponds to NMR pattern depicted in FIG. 6.

### Example 2: Preparation of Solid Form of Siponimod Adipic Acid Salt (1:1), Form P

2 g of Siponimod and 0.64 g of adipic acid were dissolved in 6 ml of methanol. The solution was diluted with 40 ml of n-heptane. 10 drops were left crystalize on a closed Petri dish at 20° C.-25° C. for 3 days to obtain crystals. The crystals were added into previously prepared solution. The mixture was stirred at 20° C.-25° C. for 75 minutes to obtain suspension. The suspension was filtered and obtained solid was washed with 5 ml n-heptane, dried (25° C./3 h/vacuum), to provide 2.105 g of Siponimod adipic acid salt (1:1), Form P. XRPD pattern of obtained solid corresponds to XRPD pattern depicted in FIG. 7, DSC pattern of obtained solid corresponds to DSC pattern depicted in FIG. 8 and NMR pattern of obtained solid corresponds to NMR pattern depicted in FIG. 9.

### Example 3: Preparation of Solid Form of Siponimod Adipic Acid Salt (1:2), Form 1

2 g of Siponimod and 1.14 g of adipic acid were mixed with 20 ml of methyl tert-butyl ether (MTBE). The suspension was stirred at 20-25° C. overnight, filtered and the filter cake was washed with 5 ml of MTBE (5 ml). Powder material was dried (25° C./1 h/vacuum) to provide 2.4 g of solid Siponimod adipic acid salt (1:2), Form 1. XRPD pattern of obtained solid corresponds to XRPD pattern depicted in FIG. 10, DSC pattern of obtained solid corresponds to DSC pattern depicted in FIG. 11 and NMR pattern of obtained solid corresponds to NMR pattern depicted in FIG. 12.

### Example 4: Preparation of Solid Form of Siponimod Adipic Acid Salt (1:0.6), Form 2

2 g of Siponimod and 0.28 g of adipic acid were mixed with 5 ml of methanol. The solution was diluted with 40 ml of n-heptane and heated to 40° C. to obtain a solution. The solution was cooled to 0° C. and stirred for 30 minutes at (−2)−0° C. The suspension was filtered, and obtained solid was washed with 2 ml n-heptane and dried (25° C./3 h/vacuum) to provide 1.8 g of Siponimod adipic acid salt (1:0.6), Form 2.

XRPD pattern of obtained solid corresponds to XRPD pattern depicted in FIG. 13, DSC pattern of obtained solid corresponds to DSC pattern depicted in FIG. 14 and NMR pattern of obtained solid corresponds to NMR pattern depicted in FIG. 15

### Example 5: Preparation of Solid Form of Siponimod Adipic Acid Salt (1:2), Form 4

0.5 g of Siponimod adipic acid salt (1:2), Form 1 was exposed to a temperature 40° C. and humidity 75% of relative humidity for between 1 month to provide 0.5 g of Siponimod adipic acid salt (1:2), Form 4. XRPD pattern of obtained solid corresponds to XRPD pattern depicted in FIG. 16.

### Example 6: Comparison Example

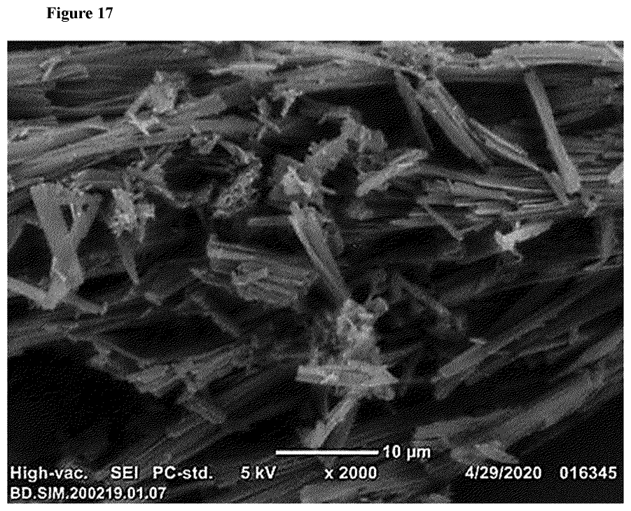

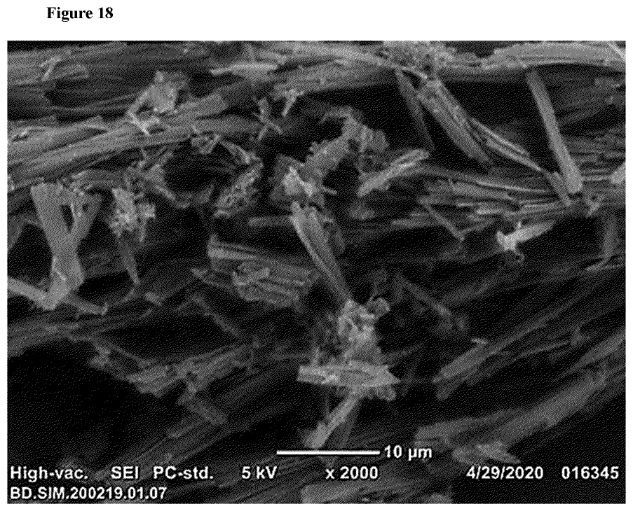

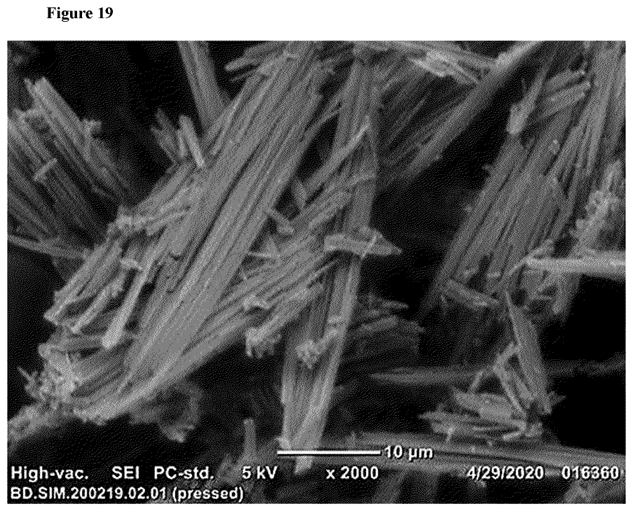

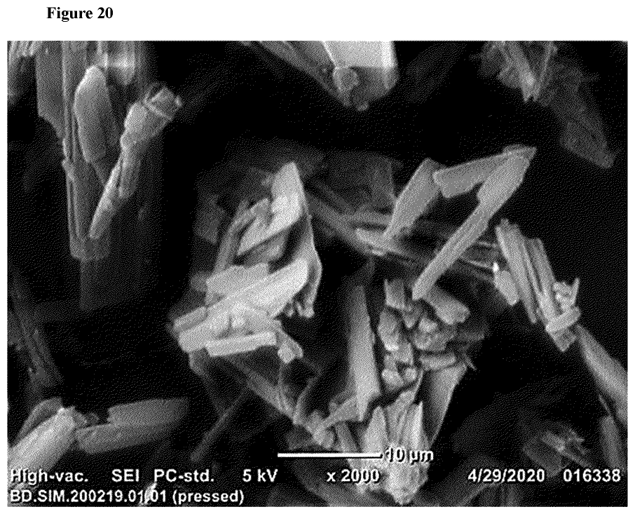

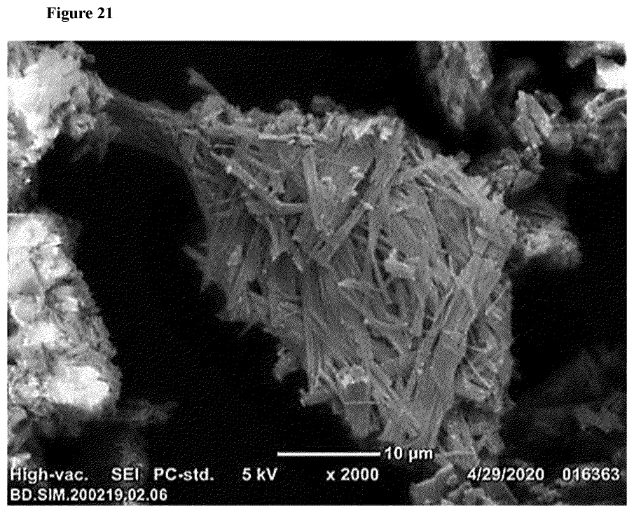

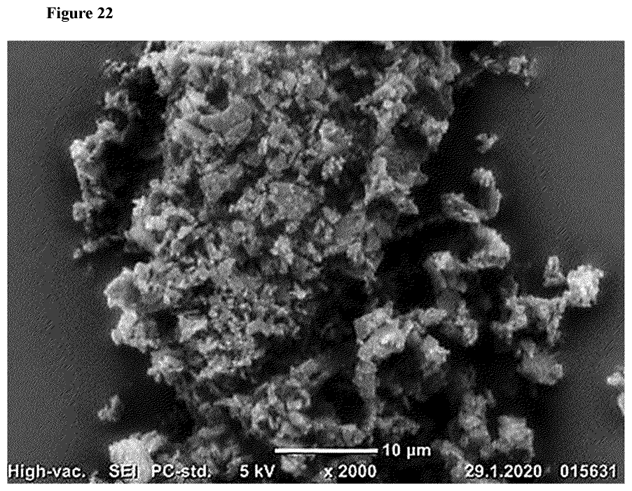

Prior art fumarate salt was prepared according to procedure described in WO2010/080409. In FIGS. 17 to 22 the crystals of prior art salt (FIG. 22) and crystals of salts of the presented invention (FIGS. 17 to 21) are depicted. The prior art salt forms small crystals that are prone to charging and processing of such crystals into a final form, for example a tablet, is therefore challenging. Contrary to that, the salts according to the presented invention form bigger crystals not prone to charging with improved processability.

### Example 7: Preparation of Solid Form 11 of Cocrystal of Siponimod with Adipic Acid (2:1)

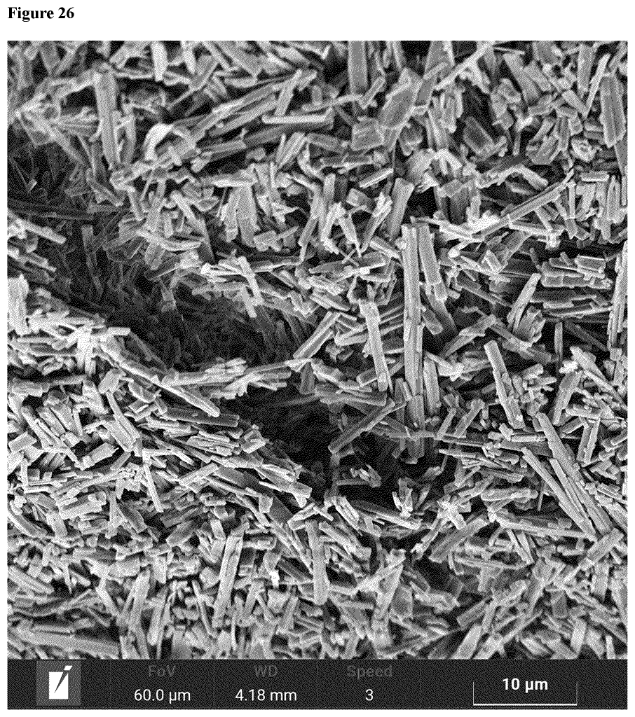

360 mg of Siponimod was dissolved in 0.6 ml of ethylacetate. To the mixture 51 mg of adipic acid was added. The mixture was sonicated for 5 minutes. Solid mass was filtered off to obtain 380 mg of solid Form 11 of cocrystal of Siponimod with adipic acid. XRPD pattern of obtained solid corresponds to XRPD pattern depicted in FIG. 23. DSC pattern of obtained solid corresponds to DSC pattern depicted in FIG. 24. The crystals of obtained solid are depicted in FIG. 26.

### Example 8: Preparation of Solid Form 11 of Cocrystal of Siponimod with Adipic Acid (2:1)

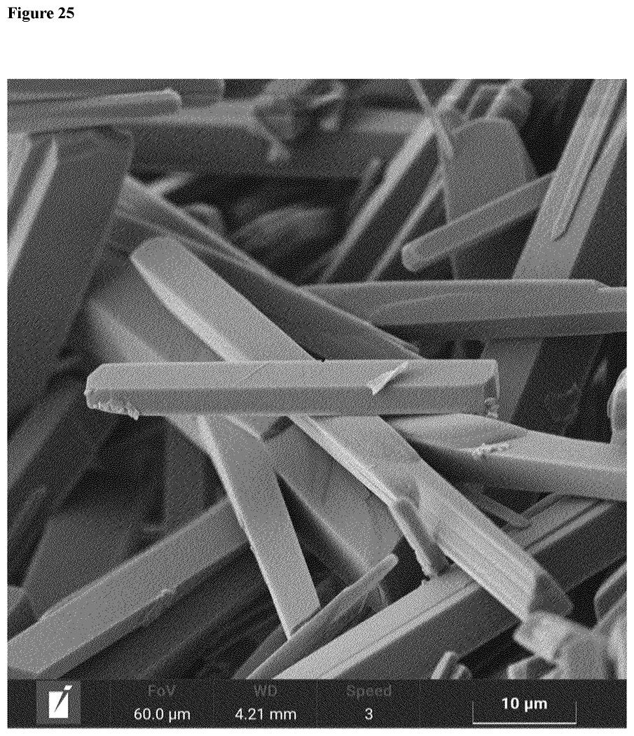

380 mg of Siponimod adipic acid adduct was mixed with 1.1 ml of ethyl acetate. The solution was heated to 70° C. and was left spontaneously cooled to 40° C. to obtain a suspension. Then it was again heated to 60° C. and cooled to ambient temperature (20-25° C.) in the course of 8 hours, decanted and the rest dried 3 h in vacuum drier at 25° C. under nitrogen to obtain 330 mg of solid Form 11 of cocrystal of Siponimod with adipic acid (2:1). XRPD pattern of obtained solid corresponds to XRPD pattern depicted in FIG. 23. DSC pattern of obtained solid corresponds to DSC pattern depicted in FIG. 24. The crystals of obtained solid are depicted in FIG. 25.

### Example 9: Preparation of Solid Form 11 of Cocrystal of Siponimod with Adipic Acid (2:1)

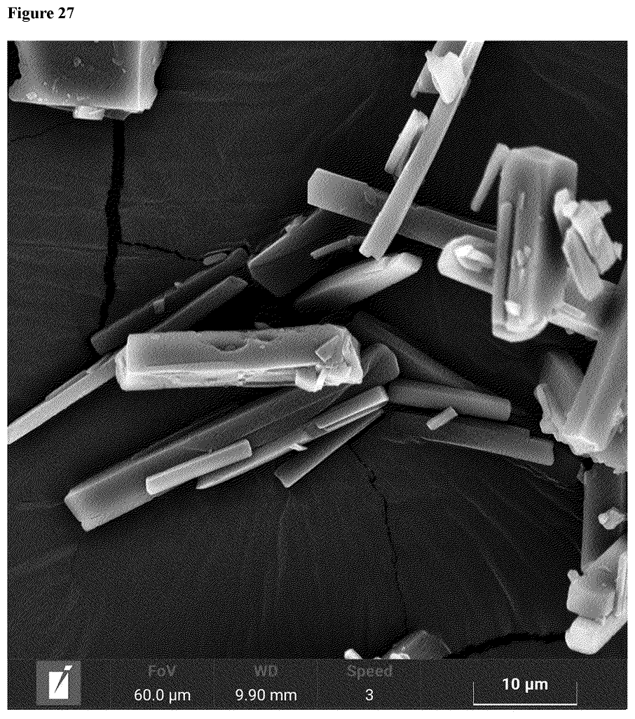

33 g of Siponimod was dissolved in 1237.5 ml of ethylacetate. The mixture was heated to 57° C. and 10.2 g of adipic acid was added. The mixture was cooled to 40° C. and stirred at this temperature for 2 hours. The mixture was cooled to 20° C. in the course of 2 hours and stirred at this temperature for 3 hours. Obtained solid was filtered off to provide 29.04 g of solid Form 11 of cocrystal of Siponimod with adipic acid (2:1) (88% of the theoretical yield), purity 99.9% (HPLC IN). XRPD pattern of obtained solid corresponds to XRPD pattern depicted in FIG. 23. DSC pattern of obtained solid corresponds to DSC pattern depicted in FIG. 24. The crystals of obtained solid are depicted in FIG. 27.

### Example 10: Preparation of Solid Form 11 of Cocrystal of Siponimod Adipic Acid (2:1), Isolation from Reaction Mixture

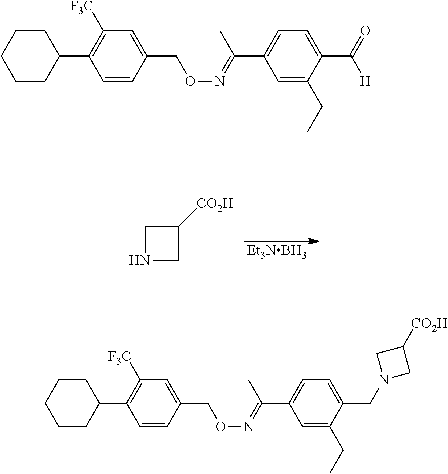

30 g of (E)-4-(1-(((4-cyclohexyl-3-(trifluoromethyl)benzyl)oxy)imino)ethyl)-2-ethylbenzaldehyde were mixed with 10.5 g of azetidine-3-carboxylic acid and 600 ml of methanol. The mixture was stirred for 30 minutes. To the mixture 7 ml of Et₃N·BH₃ complex was added in the course of 30 minutes. The mixture was heated to 30° C. and stirred at this temperature for 3 hours. The mixture was cooled to 10-15° C. and filtrated using kieselguhr. To the filtrate 750 g of water was added. The mixture was distilled off until a suspension was obtained. The mixture was cooled to 45° C. and 750 g of ethylacetate and 3 g of acetic acid were added. The phases were separated. Organic phase was filtrated using kieselguhr. To the filtrate 600 g of EtOAc was added. From the mixture 500 ml of solvent was distilled off. 500 ml of ethylacetate was added to the mixture. From the mixture 500 ml of solvent was distilled off 500 ml of ethylacetate was added to the mixture. The mixture was concentrated to the final volume 1350 ml. The rest was heated to 55° C. and 10.2 g of adipic acid were added. The mixture was cooled to 40° C. and stirred at this temperature for 2 hours. The mixture was cooled to 20° C. in the course of 2 hours. The mixture was stirred at 20° C. for 3 hours. The solid Form 11 of cocrystal of Siponimod with adipic acid (2:1) was filtered off. The yield was 85% of the theoretical yield, purity 99.9% (HPLC IN). XRPD pattern of obtained solid corresponds to XRPD pattern depicted in FIG. 23. DSC pattern of obtained solid corresponds to DSC pattern depicted in FIG. 24.

### Example 11: Comparison Example

Prior art Siponimod Fumarate (1:0.5), Form A, was prepared according to a procedure disclosed in WO2010/080409.

Water activity of the prior art form and solid forms of presented invention was measured: DVS machine ProUmid SPX-1μ Advance, 25° C., 0-90-0% relative humidity, step 10%, maximal time per step 10 hours. Equilibrium bandwidth 0.05%/60 minutes.

Water activity: a difference between the weights of the sample in 0 and 90% of relative humidity at a constant temperature. It corresponds to the amount of water taken by the tested compound when the humidity changes from 0 to 90% at a constant temperature. The higher water activity the more water can be taken by the compound and that might result in lower purity of the compound because hydrolysis impurities can be formed. It can also result in mechanical stress in final products (for example tablets) because the compound taking water increases its volume and the volume of the final product (for example tablet) is also increased and that results in cracking of the product.

In the following chart the water activities (at 25° C.) of cocrystal of Siponimod with adipic acid (2:1), Form 11 and Siponimod Fumarate (1:0.5), Form A are compared:

It can be concluded that Form 11 of cocrystal of Siponimod with adipic acid (2:1) shows lower water activity than Siponimod fumarate, Form A.

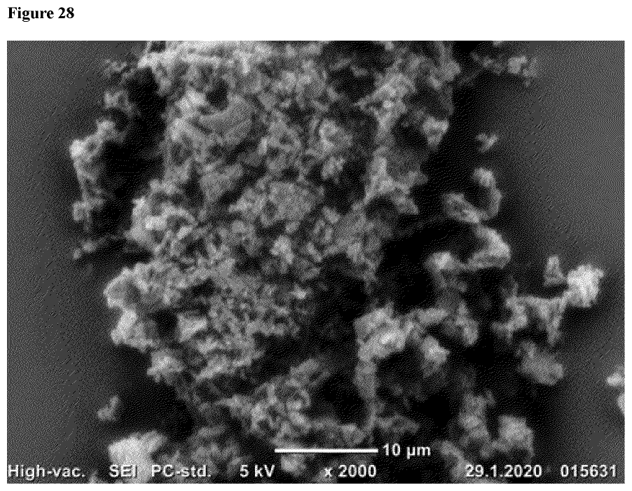

The crystals of solid Form 11 of cocrystal of Siponimod with adipic acid (2:1) are depicted in FIGS. 25 to 27. The crystal of Siponimod fumarate, Form A, are depicted in FIG. 28. It can be concluded that crystals of Form 11 are much bigger than crystals of Form A and are less prone to charging and have improved properties with respect to processability.

1. A salt or a cocrystal of Siponimod with adipic acid.

2. A solid form of a salt or a cocrystal of Siponimod with adipic acid.

3. The solid form according to claim 2, wherein said Siponimod with adipic acid is a cocrystal of Siponimod with adipic acid (2:1), Form 11, characterized by XRPD pattern having 2θ values 6.7°, 12.7° and 13.5° 2θ (±0.2 degrees 2θ).

4. The solid form according to claim 3, wherein said cocrystal is characterized by XRPD pattern having 2θ values 6.7°, 7.5°, 12.7°, 13.5°, 16.4° and 18.3° 2θ (±0.2 degrees 2θ).

5. A process for preparation of the solid form according to claim 3 comprising:
a) dissolving Siponimod in ethylacetate;
b) adding adipic acid; and
c) isolating the solid form.

6. The process according to claim 5 wherein ethylacetate comprises up to 3% (vol %) of water.

7. The solid form according to claim 2, wherein said Siponimod with adipic acid is a salt of Siponimod with adipic acid (1:0.6), Form 2, characterized by XRPD pattern having 2θ values 4.0°, 7.4°, 17.8° and 19.3° 2θ (±0.2 degrees 2θ).

8. A process for preparation of the solid form according to claim 7 comprising:
a) dissolving Siponimod and adipic acid in methanol;
b) adding n-heptane; and
c) isolating the solid form.

9. The solid form according to claim 2, wherein said Siponimod with adipic acid is a salt of Siponimod with adipic acid (1:2), Form 1, characterized by XRPD pattern having 2θ values 15.5°, 18.0°, 19.3 and 21.5° 2θ (±0.2 degrees 2θ).

10. A process for preparation of the solid form according to claim 9 comprising:
a) suspending Siponimod and adipic acid in methyl tert-butyl ether; and
b) isolating the solid form.

11. The solid form according to claim 2, wherein said Siponimod with adipic acid is a salt of Siponimod with adipic acid (1:1), Form P, characterized by XRPD pattern having 2θ values 5.6°, 7.5° and 19.2° 2θ (±0.2 degrees 2θ).

12. A process for preparation of the solid form according to claim 11 comprising:
a) dissolving Siponimod and adipic acid in methanol and n-heptane; and
b) isolating the solid form.

13. The solid form according to claim 2, wherein said Siponimod with adipic acid is a salt of Siponimod with adipic acid (1:2), Form Q′, characterized by XRPD pattern having 2θ values 5.5°, 17.5° and 18.8° 2θ (±0.2 degrees 2θ).

14. A process for preparation of the solid form according to claim 12 comprising:
a) dissolving Siponimod and adipic acid in methyl tert-butyl ether;
b) isolating a solid form; and
c) contacting the solid with humidity.

15. The process according to claim 14 wherein the humidity is between 80% and 100% RH.
